(19) United States
(12) Patent Application Publication
(10) Pub. No.: US 2011/0313462 A1
(43) Pub. Date: Dec. 22, 2011

(54) CONTROL OF INNATE SYSTEMS TO REMODEL SPINAL CANAL CROSS-SECTIONAL AREA

(76) Inventor: Neville Alleyne, Oceanside, CA (US)

(21) Appl. No.: 12/817,020

(22) Filed: Jun. 16, 2010

(51) Int. Cl. A61B 17/88

(52) U.S. Cl. 606/279

(57) ABSTRACT

Retracting a resected dorsal portion of a vertabra from a disk bearing ventral portion whereby the spinal canal cross section area of the vertabra is increased and then fixing the separated vertabra portions together while maintaing a gap thereby maintaining the larger cross sectional area.

### FIELD OF THE DISCLOSURE

This disclosure relates generally to neurosurgical devices and procedures to reconfigure portions of the spinal canal.

### BACKGROUND

Medical and physiological conditions such as acquired central and neural foraminal stenosis which are found in the population can be debilitating. It has been estimated by some that by the year 2030, approximately 45% to 50% of the population will be 65 or greater and with increased population age the condition of spinal stenosis and its clinical presentation of neurogenic claudication may continue to grow. Traditional treatment options include performing decompressive laminectomies, partial medial facetectomies, and/or partial foraminotomies in order to gain adequate decompression. Such procedures tend to result in some level of iatrogenic instability into the motion segments involved.

In the case of spinal stenosis pressure (compression) on the nerve, dura or thecal sac can lead to pain and discomfort which may limit walking, bending and deteriorate the quality of life. Pain and discomfort may also lead to the use of and narcotics, anti-inflammatory medications, muscle relaxers, epidural steroid injections, and physical therapy.

### SUMMARY

In some exemplary implementations there is at least one of a surgical procedure, tool, system and method whereby the cross-sectional area within a selected region of the spinal canal is increased.

In some exemplary implementations there is at least one of a surgical procedure, tool, system and method whereby directed innate spinal bone growth increases the cross-sectional area within a selected region of the spinal canal.

In some exemplary implementations there is at least one of a surgical procedure, tool, system and method whereby a part of the spinal canal is re-engineered to increase the cross-sectional area within a region of the spinal canal. In some instances bones bounding the spinal canal such as the pedicles are extended to form said increased cross section. In some instances the extension is via fixtures. In some instances the extension is via a substrate such as a bone promoting material. In some instances resected pedicles fixed with screws are then packed with bone promoting material. Optionally, an interior shield may be added (in some cases depending on the severity of the spinal canal narrowing) to the boney area bounding the spinal canal. Such a shield has the properties of discouraging bone growth into the spinal canal.

In some exemplary implementations there is at least one of a surgical procedure, tool, system and method whereby a part of the spinal canal is altered or “remodeled” via controlled innate bone growth to increase the cross-sectional area at a selected point or points. In some instances the increase in area is initially stabilized by one or more fixtures forming a pre-determined distance between edges of resected vertebral bone.

In some exemplary implementations there is at least one of a surgical procedure, tool, system and method whereby a part of the spinal canal is altered or “remodeled” via controlled innate bone growth to increase the cross-sectional area at a selected point or points. In some instances the increase in area is initially stabilized by one or more fixtures forming a pre-determined distance between edges of resected vertebral bone. In some instance bone promoting material may be incorporated into or with the fixtures.

In some exemplary implementations there is disclosed a method of forming fixing channels in spinal bone bordering the spinal canal, resecting spinal bone on either side of a vertebra, forming a gap between ends of resected spinal bone thereby increasing a cross sectional area of a plane measurement of the spinal canal which has for boundaries the resected bone and fixing said spinal bone to maintain the increased cross section. In some instances bone promoting material is packed, added or placed into the gap area. In some instances an interior shield is placed within the spinal canal to discourage bone growth into the spinal canal.

In some exemplary implementations the method of increasing the cross sectional area of a portion of a spinal canal wherein pedicles on either side of a vertebra are drilled to form channels for fixing fasteners such as threaded screws. Said pedicles are thereafter resected, a gap between the ends of resected pedicles is formed and maintained with one or more screw fixtures inserted into the drilled channels. In some instances bone promoting material is packed, added or placed into the gap area. In some instances an interior shield is placed within the spinal canal to discourage bone growth into the spinal canal. In some instances a gap spacer is also a bone promoting material. In some instances a gap spacer is fixed to an interior shield. In some instances a gap spacer is fixed to an exterior shield.

In at least one exemplary implementation, or an aspect thereof, disclosed are orthopedic and neurosurgical devices and procedures to reconfigure portions of the spinal canal and neural foramen. By increasing the cross-sectional diameter of the spinal canal or neural foramen, compression on the thecal sac or nerve roots can be diminished. If some compression can be diminished, the symptoms within the patient's leg may diminish and obviate the need for more complex open procedures. Performing remodeling of the spinal canal or neural foramen through minimally invasive approaches reduces trauma. In some instances removal of a section of the dorsal element which may include spinous process, lamina, or pars can be used to increase the cross-sectional diameter. Reattachment of the bone can be facilitated via guides and screws or fasteners such as cannulated and fenestrated screws which may allow bone graft materials such as (but not limited to) DBM, autogenous bone graft, allograft, and BMP (bone morphogenetic proteins such as INFUSE or PMMA) to be included.

In addition to reattachment of spinous process and lamina or removal of dorsal element and reshaping, placing pedicle screws for reattachment then can be modified by removing bone from the ventral surface of the posterior element to increase cross-sectional diameter or add an additional gap between the posterior element reattachment so that bone can fill in at the osteointegration site and allow for greater cross-sectional diameter of the thecal sac.

Other features and advantages of the present disclosure will be set forth, in part, in the descriptions which follow and the accompanying drawings, wherein the implementations of the present disclosure are described and shown, and in part, will become apparent to those skilled in the art upon examination of the following description taken in conjunction with the accompanying drawings or may be learned by practice of the present disclosure.

### DRAWINGS

FIG. 1 is an illustration of a vertebra in the lumbar region.

FIG. 2 is an illustration of a vertebra in the lumbar region with narrowing of the canal.

FIGS. 3A through 3C are sequential views of a vertebra with increased spinal canal cross section.

FIG. 4 is an illustration of a resected vertebra with fasteners.

FIGS. 5A, 5B and 5C are views of a resected vertebra with fasteners and a component view of the canal shield.

FIG. 6 is an illustration of a resected vertebra with fastener.

FIG. 7 is an illustration of a vertebra and nerves with resecting shield.

### ADDITIONAL DETAILS

Microdissection means a cut or stab incision which has the following characteristics the incision is generally less than 14 mm.

Re-engineering and/or rearchitecting the spine refers to modifying existing structural elements of the spine (without complete removal) to provide more space in which at least one of nerves, thecal sac and dura reside.

Neural elements , depending on the level being addressed, includes dura, nerves, arachnoid, pia, epidural space, subdural cavity, sub arachnoid cavity, thecal sac, and all associated functional and structural elements.

Innate bone growth means the body's innate capacity for bone formation in response to tension forces on the repair callus.

Bone promoting material includes, but is not limited to, one or more (used separately or in combination) of autogenous bone grafts, allograft bone graft, platelet gels, bone-morphogenetic proteins powerful proteins and glycans that are stimulants for bone formation, demineralized bone matrix (DBM),wherein an allograft bone is demineralized to extract proteins that stimulate bone formation are extracted from the bone, and ceramics porous and mesh combined with collagen or other growth promoting proteins.

In the following description, various exemplary implementations, aspects and characteristics are discussed as directed toward surgical instruments, tools, systems and methods more particularly applied to the spine. The focus on this application is not intended to be, nor should it act as, a limitation to the scope of this disclosure. The other features and advantages of the present disclosure will be set forth, in part, in the descriptions which follow and the accompanying drawings, wherein the implementations of the present disclosure are described and shown, and in part, will become apparent to those skilled in the art upon examination of the following description taken in conjunction with the accompanying drawings or may be learned by practice of the present disclosure.

Heading and Titles are not intended to be limitations and should be read in a general sense. Implementations may include several novel features, no single one of which is solely responsible for its desirable attributes or which is essential to practicing the disclosure described herein. The advantages of the present disclosure may be attained by means of the instrumentalities and combinations particularly pointed out in the disclosure and any appended claims.

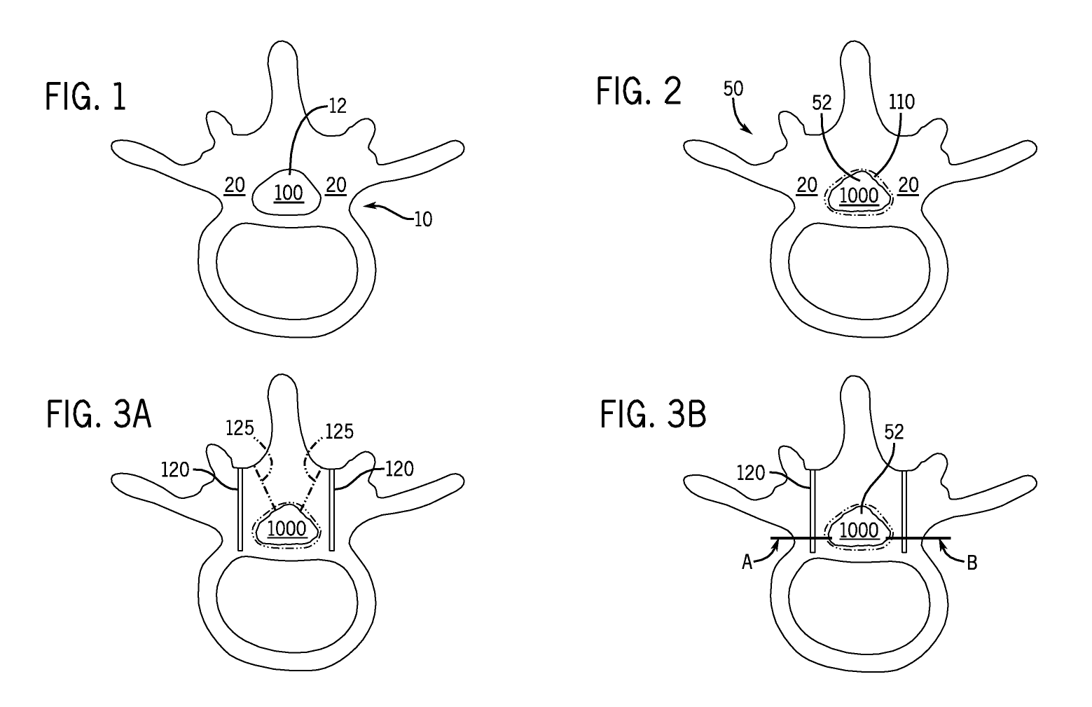

FIG. 1 shows a downward view of a cross section of a lumbar vertebra of the human body. It will be understood by those of ordinary skill in the art that the specific illustration of lumbar vertebra or the discussion of the lumbar region is not a limitation. Appling the instant disclosure to the thoracic and cervical spinal regions of the spine are all within the intended scope of this disclosure. Vertebra 10 surround or forms the spinal canal 12 with a cross sectional area “CS^{A1}” 100. As shown in FIGS. 1 and 7 pedicles 20 are formed as part of vertebra 10 on either side of the spinal canal 100.

FIG. 2 shows another vertebra 50 and in this illustration the spinal canal 52 also as a cross sectional area “CS^{A2}” 1000. For purposes of illustration the vertebra shown in FIG. 1 and FIG. 2 are the same size. It is apparent that the cross sectional area “CS^{A1}” 100 of FIG. 1 is larger than the cross sectional area “CS^{A2}” 1000 of FIG. 2 the reduction in area (shown in the FIG. 2 as reduction region 110 surrounding the spinal canal) is often caused by a condition known as spinal stenosis (derived from the Greek “stenos”) in which a person has narrowing of the spinal canal. The narrowed canal can place pressure on neural elements from the dura inward to the nerves or on the nerves that branch off the spinal cord. Spinal stenosis can occur anywhere in the spine, but most cases of spinal stenosis occur in the lower back. The most common cause of spinal stenosis is osteoarthritis. The most common symptoms of spinal stenosis include neck pain or back pain. Additional symptoms may include shoulder pain, arm pain, arm numbness, arm weakness, leg pain, leg numbness, leg weakness, impotence, or urinary incontinence. Treatment for spinal stenosis may include rest, a neck or back brace, physical therapy, corticosteroid medication, or nonsteroidal anti-inflammatory medications for pain. Additional treatment may include surgery.

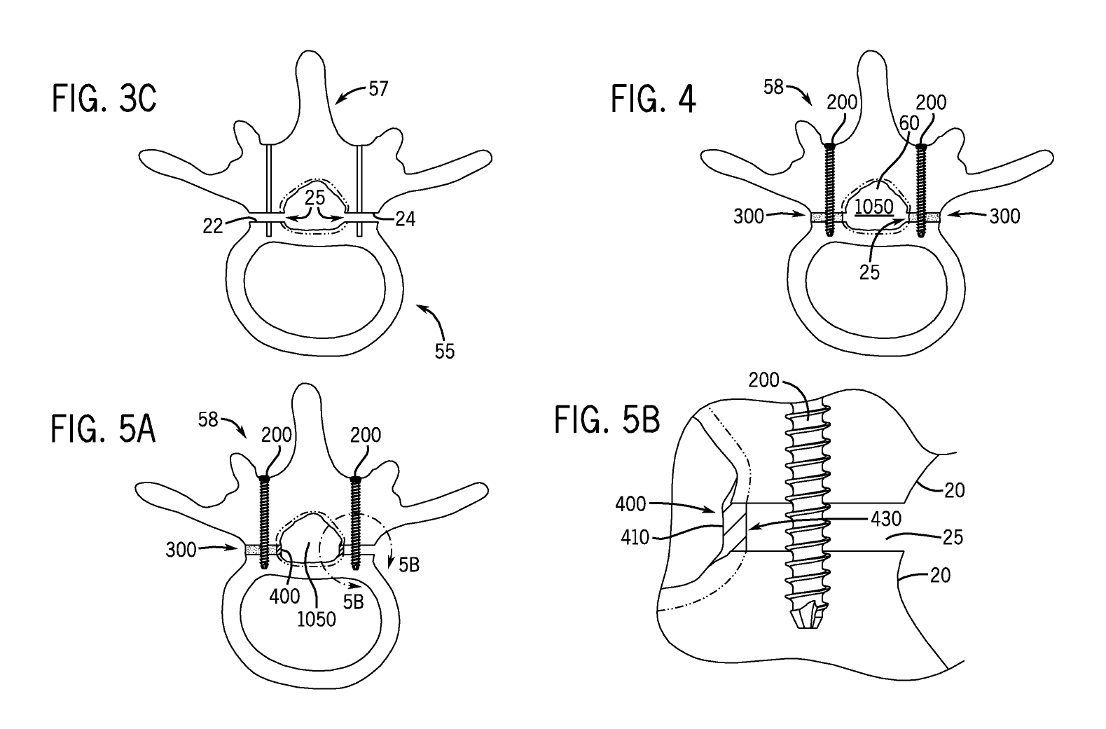

In FIG. 3A fixing channels 120 formed in pedicles 20 from the dorsal side inward, prior to the resection and retraction of the resected vertebra are illustrated. Those of ordinary skill in the art will recognize that fixing channels may also be formed in nearby areas. Dotted lines 125 indicate a different location for said fixing channels within the dorsal region of a vertebra. Retraction should keep the fixing channels in line between the regions of the cut vertebra. The call out of the pedicles as the region on the dorsal side of the vertebra having the fixing channel is not a limitation and depending on the location(s) in the spinal canal of the reduction region its morphology and the morphology of the neural elements of the patient, the resection may or may not be limited to the pedicles but may include or be of, spinous process and lamina (unilaterally or bilaterally). The pars may be identified and cut as indicated with fixing pars channels 125 and the posterior elements removed which include the spinous process and lamina, as well as the pars. The pars is then reshaped and fibrous tissue removed. A screw which may be cannulated is then reinserted to expand a variable gap to improve cross-sectional diameter in the neural foramen. Cannulated crews are uniquely designed to allow bone graft.

Prior to cutting the screw holes, a K-wire may be placed down the pedicle to maintain orientation. The K-wire is confirmed on x-ray, AP and lateral fluoroscopic images, to be in good position. The K-wire can be left in place and the screw can then be placed over it. The K-wire may also be removed.

In the and FIGS. 3B and 3C show a vertebra with a narrowed spinal canal 52 is resected at pedicles 20 along line “A” and line “B”. Retracting or separating the resected region of the vertebra provides a disc bearing ventral region 55 and a dorsal region 57. The disc bearing ventral region 55 includes a first half 22 of the pedicles 20 with terminal surfaces 22 corresponding to the resection. The dorsal region includes a second half 24 of the pedicles 20 with terminal surfaces 24 corresponding to the resection. A gap 25 is shown bordered by, or between the two halves illustrative of an area of resected bone and tissue. The vertebra region containing the disc may also be referred to as the larger vertebral body. It is critical that neural elements within the spinal canal 52 or extending from the spinal canal (see FIG. 7) not be damaged during the forming of the fixing channel or the resection.

Shown in FIG. 4 is a rearchitected vertebra 58. The rearchitected vertebra used in the restructuring are an exemplary implementation in which resected pedicles 20 are separated and a gap is maintained, rather than bringing the terminal surfaces 22 and 24 together, by fixing the pedicles together with a fixing fastener such as the threaded bone screw 200 inserted into fixing channels 120. It is preferred that the separated pedicles reattach by bone growth thereby fusing the two portions or regions of the vertebra together while marinating the larger spinal canal. To facilitate such bone growth a biocompatible bone promoting material 300 may be add and/or packed into the gaps 25.

Calling out pedicles as the bone portion of the vertebra being resected to enlarge the cross sectional area of the spinal canal is not a limitation. Those or ordinary skill will recognize that in some instance resection of additional or different portions of the bone in the dorsal region, such as lamina may be indicated.

The bone screws 200 may also be configured to promote bone growth. In some instances the bone screws have a modified surface. Such a surface may be roughened. It is well known in the art to use sintering, etching and coating as some of the ways to provide a surface roughness. A 2-8 micron range of a peak to valley is preferred with a more preferred range being 3-5 microns peak to valley. Materials for such bone screws 200 include but are not limited to titanium, stainless steel, cobalt chrome molybdenum, PMMA, PLA, PGA, PLLA , nitinol, tantalum, carbon, polymers, plastics , ceramic and PEEK or compositions containing two or more of same. Screws may be solid body or fenestrated. In some instance when the screws are constructed of PLA, PGA or PLLA they may be bioabsorbable thereby eliminating the need to remove such screws in the event removal is appropriate.

The fixed together pedicles 20 expand the spinal canal from the narrowed canal 1000 with cross sectional area “CS^{A2}” 1000 to an enlarged canal 60 with a cross sectional area “CS^{A3}” 1050. The larger cross sectional diameter spinal canal may relieve pressure on neural elements therein.

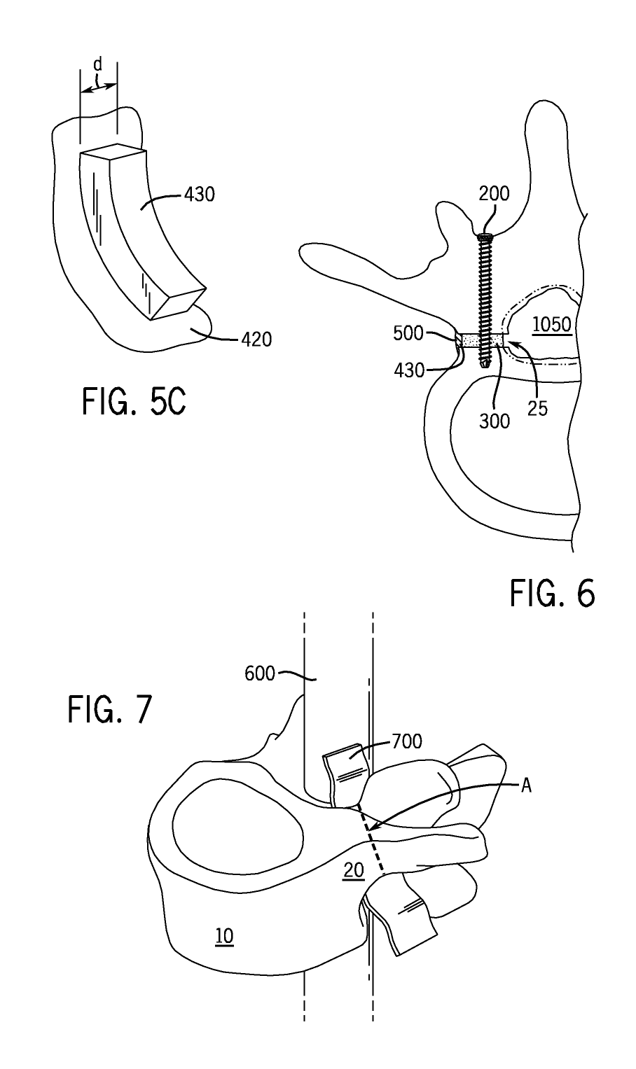

Shown in FIGS. 5A and 5B is an exemplary implementation of another resected vertebra in which pedicles 20 have been cut through and fixed via screws 200. To promote bone growth, to fill in the gap, directed away from the spinal canal 1050 interior shield 400 is inserted in the gap 25. The shield has a canal facing surface 410, a bone facing surface 420 that is placed against the bone structure of the spinal canal. Gap spacer 430 may be of a pre-selected diameter “d” to maintain a designated gap between the resected ends of the pedicles. A variety of such shields may be provided during a surgery to allow the surgeon the option of selected in the proper size and type of shield.

Those of ordinary skill in the art will recognize that the shield 400 and gap spacer 430 should be bio-compatible. The gap spacer maintains a predetermined gap. The gap spacer may also acts at least a portion of the bone growth promoting system of the disclosure. The gap spacer (which also may be inserted into the gap without connection to a shield—not shown) can serve a bone encouragement and/or promotion function. For the gap spacer 430 to participate in at least one of the promotion, direction and encouragement of bone growth it should be constructed of, enhanced with, or coated with bone promoting material with at least one of surface roughness and porosity which presents an outer surface for coating with bone growth and/or pores that form a scaffolding for bone growth within gap spacer 430.

The gap spacer preferably is attached to or formed as part of the shield 400. When interior shield 400 is inserted in a spinal canal it should have the property of the canal facing surface 410 being of a configuration and material that is both biocompatible and discouraging bone growth thereon. Said canal facing surface 410 acts as a barrier to discourage bone growth into the spinal canal. Encouraging bone growth into the spinal canal is contrary to expanding the cross sectional area of the spinal canal and should be avoided as much as practical.

The bone facing surface 420 that is placed against the bone structure of the spinal canal may be bone growth inert (not configured to promote bone growth) or it may also be constructed of a bone growth promoting material or it may have a bone promoting surface.

Interior shield 400 may have discreet regions each of which may have different bone promoting properties. In addition to having a tool kit of shields and screws of varying sizes and diameters. The shields may also be provided in different region configurations dependant on the circumstance of the usage.

In addition to the screws and/or gap spacer being used to maintain gap and encourage or promote the innate response of the body to grow spinal bone, as shown in FIG. 6 an exterior shield 500 with an additional gap spacer 430 may be used to direct, promote or encourage bone growth within the gap. Bone growth extending beyond the resected region of pedicles 20 outside the spinal canal should also be minimized when practicable.

In FIG. 7 a vertebra 10 and the neural elements shown as the dura 600 and including all inner and outer associated elements. A malleable retractor can be placed along the medial wall of the pedicle to prevent inadvertent injury to the nerve root or thecas sac during the resection or cutting. A similar retractor can also be placed on the lateral aspect of the pedicle to prevent injury to the exiting nerve root. The inferior and superior aspect of the pedicle should be more easily cut because visualization and retraction of the thecal sac and adjacent nerve roots can be more easily protected with traditional retractors.

Prior to resecting pedicles 20 along line “A” and “B” a removable barricade 700 should be inserted between the pedicle and the neural element 600 to limit the impact of the resecting on the surrounding tissue and in particular on the neural elements.

While the method and agent have been described in terms of what are presently considered to be the most practical and preferred implementations, it is to be understood that the disclosure need not be limited to the disclosed implementations. It is intended to cover various modifications and similar arrangements included within the spirit and scope of the claims, the scope of which should be accorded the broadest interpretation so as to encompass all such modifications and similar structures. The present disclosure includes any and all implementations of the following claims.

It should also be understood that a variety of changes may be made without departing from the essence of the disclosure. Such changes are also implicitly included in the description. They still fall within the scope of this disclosure. It should be understood that this disclosure is intended to yield a patent covering numerous aspects both independently and as an overall. system and in both method and apparatus modes.

Further, each of the various elements of the disclosure and claims may also be achieved in a variety of manners. This disclosure should be understood to encompass each such variation, be it a variation of an implementation of any apparatus implementation, a method or process implementation, or even merely a variation of any element of these.

Particularly, it should be understood that as the disclosure relates to elements of the implementation, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this disclosure is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting , such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternatives.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

All callouts associated with figures are hereby incorporated by this reference.

Since certain changes may be made in the above apparatus without departing from the scope of the invention herein involved, it is intended that all matter contained in the above description, as shown in the accompanying drawing, shall be interpreted in an illustrative, and not a limiting sense.

What is claimed:

1. A procedure to increase a cross sectional area of a spinal canal comprising:
forming at least one fixing channels through a portion of the dorsal side of a vertebra;
resecting a portion of said dorsal side of said vertebra to divide the vertebra into a disk bearing ventral region and a dorsal region;
forming a gap between the bone on either side of said resection and retracting the dorsal region; and, inserting fixtures into said fixing channel to connect the vertebra while maintaining said gap.

2. The procedure of claim 1 wherein the fixtures are screws with a bone promoting material applied thereon.

3. The procedure of claim 2 wherein the fixtures are screws are fenestrated.

4. The procedure of claim 1 wherein one or more gap spacers is positioned within the said gap.

5. The procedure of claim 4 wherein said gap spacers are formed of a bone promoting material.

6. The procedure of claim 1 wherein said gap is filled with bone promoting material.

7. The procedure of claim 1 further comprising fixing an interior shield to the resected bone in the spinal canal.

8. The procedure of claim 1 wherein the interior shield discourages bone growth extending from said gap into said spinal canal.

9. The procedure of claim 5 further comprising an interior shield which discourages bone growth in combination with said gap spacers.

10. A method to increase a cross sectional area of a spinal canal the method comprising:
forming a fixing channel through each pedicles on a vertebra;
resecting each of said pedicles to divide the vertebra into a disk bearing ventral region and a dorsal region;
forming a gap between the ends of said resected pedicles by retracting the dorsal region; and,
inserting fixtures into the fixing channel of each of said pedicle while maintaining said gap.

11. The method of claim 10 wherein the fixtures are screws with a bone promoting material applied thereon.

12. The method of claim 10 wherein the fixtures are screws are fenestrated.

13. The method of claim 10 wherein one or more bone promoting material gap spacers are positioned within the said gap.

14. The procedure of claim 10 wherein said gap is filled with bone promoting material.

15. The method of claim 8 the method further comprising fixing an interior shield to the resected pedicles in the spinal canal.

16. The procedure of claim 10 wherein the interior shield discourages bone growth extending from said gap into said spinal canal.

17. A method to change a dimension of the spinal canal within a vertebra, the method comprising:
forming fixing channels between the larger vertebral body and a portion of the dorsal region of the vertebra;
resecting said dorsal region of a vertebra; and,
reattaching said dorsal region to said larger vertebral body while maintaining a gap between at least a portion of said dorsal region and said larger vertebral body

18. The method of claim 17, wherein said reattaching is provided via screws in said fixing channels.

19. The method of claim 18 wherein said screws are fenestrated.
